(19) United States
(12) Patent Application Publication
(10) Pub. No.: US 2020/0367884 A1
(43) Pub. Date: Nov. 26, 2020

(54) SURGICAL INSTRUMENT SLEEVE SYSTEM

(71) Applicants: Israt Nisa, Toledo, OH (US); The University of Toledo, Toledo, OH (US)

(72) Inventors: Mohammad Mahbub Jamil, Toledo, OH (US); Israt Nisa, Toledo, OH (US)

(73) Assignee: The University of Toledo, Toledo, OH (US)

(21) Appl. No.: 16/878,684

(22) Filed: May 20, 2020

Related U.S. Application Data
(60) Provisional application No. 62/850,716, filed on May 21, 2019.

(51) Int. Cl. A61B 17/04

(52) U.S. Cl. CPC A61B 17/0493; A61B 2017/06028; A61B 17/0483; A61B 17/0466

(57) ABSTRACT

A surgical instrument sleeve system and method of using the same are described.

### RELATED APPLICATIONS

This application claims priority to U.S. Provisional Application No. 62/850,716 filed under 35 U.S.C. § 111(b) on May 21, 2019, the disclosure of which is incorporated herein by reference in its entirety.

### STATEMENT REGARDING FEDERALLY SPONSORED RESEARCH

This invention was made with no government support. The government has no rights in this invention.

### BACKGROUND

During certain surgical procedures, a surgeon may hold a needle driver with his or her dominant hand and a pair of forceps with his or her nondominant hand. The surgeon uses the needle driver to hold a needle and to pass the needle through the tissue. Suture material may be attached to the needle and may be very fine. The surgeon uses the forceps to manipulate tissue and to align the tissue for passage of the needle. The forceps may also be used as a support to adjust the position of the needle in the needle driver. During the suturing portion of surgical procedures, the suture line is generally held tight to prevent the suture from unraveling. However, the instruments currently held by the surgeon during surgical procedures prevent direct handling of the suture by the surgeon. Due to the delicate nature of the suture material, neither the forceps nor the needle driver can be used to manipulate the suture. Thus, either the surgeon must put down the forceps to handle the suture, or a surgical assistant has to handle the suture for the surgeon. Putting down the forceps creates an additional step for the surgeon. And, a capable surgical assistant may not always be available to apply traction to the suture to prevent the suture line from unraveling. In addition, suture handling is also limited in deep body cavities where space is limited and the assistant's hands may not readily reach. It would therefore be desirable to improve the function of surgical instruments, such as forceps, for the dynamic handling of delicate suture material.

Moreover, economy of motion is important in surgery. The surgeon often needs to change the angle of the needle in the jaws of the needle driver. A surgeon may accomplish this by first disengaging the needle completely and then re-engaging the needle in the needle driver. Or, in the alternative, the grip on the needle by the needle driver may be loosened and the angle may be changed by pushing the needle against tissue or forceps held in the surgeon's non-dominant hand. However, the surgeon must be careful not to touch the tissue or the forceps with the sharp tip of the needle because the tip can lacerate delicate tissue, or the tip can be damaged by contact with the metal forceps. Thus, there is a need to overcome the limitations of existing surgical instruments, such as forceps, in handling suture material in a way that allows a surgeon to manipulate fine suture material without releasing the surgical instrument and which allows the surgeon to manipulate sutures in deeper body cavities where access may be limited. Moreover, there is a need to provide a surface that can be used as a support by the surgeon to change the angle of the needle.

### SUMMARY

Provided herein is a surgical instrument sleeve system comprising a first sleeve having a first sleeve body, wherein the first sleeve body includes a first sleeve bore extending within the first sleeve body between a first sleeve first opening at a first sleeve first end and a first sleeve second opening at a first sleeve second end; a second sleeve having a second sleeve body, wherein the second sleeve body includes a second sleeve bore extending within the second sleeve body between a second sleeve first opening at a second sleeve first end and a second sleeve second opening at a second sleeve second end; and a bridging member connecting the first sleeve and the second sleeve.

In certain embodiments, the first sleeve body is cylindrical. In particular embodiments, the second sleeve body is also cylindrical. In certain embodiments, the first sleeve has a first sleeve engagement surface, the second sleeve has a second sleeve engagement surface, and the first sleeve engagement surface is configured to cooperate with the second sleeve engagement surface.

In certain embodiments, the first sleeve further includes a first magnetic core positioned between the first sleeve body and the first sleeve bore. In particular embodiments, the second sleeve further includes a second magnetic core positioned between the second sleeve body and the second sleeve bore. In particular embodiments, the first magnetic core is concentric with the first sleeve body and the first sleeve bore, and the second magnetic core is concentric with the second sleeve body and the second sleeve bore. In particular embodiments, the first magnetic core is concentric with only one but not both of the first sleeve body but not the first sleeve bore. In particular embodiments, the second magnetic core is concentric with only one but not both of the second sleeve body and the second sleeve bore.

In certain embodiments, the first sleeve and the second sleeve are formed from a surgical quality resilient material. In particular embodiments, the surgical quality resilient material comprises a medical-grade plastic, foam, or rubber.

In certain embodiments, the bridging member is formed integrally with the first sleeve and the second sleeve. In certain embodiments, the bridging member is arcuate. In certain embodiments, the bridging member includes a bridging member engagement surface. In particular embodiments, the bridging member engagement surface is serrated. In certain embodiments, the bridging member has a bridging member first end attached to the first sleeve at the first sleeve second end and a bridging member second end attached to the second sleeve at the second sleeve second end. In certain embodiments, the bridging member is formed from a flexible material.

Further provided herein is a surgical instrument sleeve system comprising a first sleeve having a first sleeve body and a first magnetic core, wherein the first sleeve body includes a first sleeve bore extending within the first sleeve body between a first sleeve first opening at a first sleeve first end and a first sleeve second opening at a first sleeve second end, and wherein the first magnetic core is positioned between the first sleeve body and the first sleeve bore; and a second sleeve having a second sleeve body and a second magnetic core, wherein the second sleeve body includes a second sleeve bore extending within the second sleeve body between a second sleeve first opening at a second sleeve first end and a second sleeve second opening at a second sleeve second end, and wherein the second magnetic core is positioned between the second sleeve body and the second sleeve bore.

In certain embodiments, the first sleeve body is cylindrical. In particular embodiments, the second sleeve body is also cylindrical.

In certain embodiments, the first sleeve has a first sleeve engagement surface, the second sleeve has a second sleeve engagement surface, and the first sleeve engagement surface cooperates with the second sleeve engagement surface.

In certain embodiments, the first sleeve and the second sleeve are formed from a surgical quality resilient material. In particular embodiments, the surgical quality resilient material includes a medical-grade plastic, foam, or rubber.

In certain embodiments, the surgical instrument sleeve system further includes a bridging member connecting the first sleeve and the second sleeve. In particular embodiments, the bridging member is formed integrally with the first sleeve and second sleeve. In particular embodiments, the bridging member includes a bridging member engagement surface. In particular embodiments, the bridging member engagement surface is serrated. In particular embodiments, the bridging member has a bridging member first end attached to the first sleeve at the first sleeve second end and a bridging member second end attached to the second sleeve at the second sleeve second end. In particular embodiments, the bridging member is arcuate.

Further provided herein is a method of using a surgical instrument sleeve system comprising attaching a first sleeve to a first jaw of a surgical instrument, wherein the first sleeve has a first sleeve body, and wherein the first sleeve body includes a first sleeve bore extending within the first sleeve body between a first sleeve first opening at a first sleeve first end and a first sleeve second opening at a first sleeve second end; attaching a second sleeve to a second jaw of a surgical instrument, wherein the second sleeve has a second sleeve body, wherein the second sleeve body includes a second sleeve bore extending within the second sleeve body between a second sleeve first opening at a second sleeve first end and a second sleeve second opening at a second sleeve second end; positioning the first sleeve at a location on the first jaw and the second sleeve at a location on the second jaw such that a first sleeve engagement surface on the first sleeve cooperates with a second sleeve engagement surface on the second sleeve; and handling suture material between the first sleeve engagement surface and the second sleeve engagement surface.

In certain embodiments, the first sleeve and the second sleeve are connected with a bridging member. In particular embodiments, the bridging member is formed integrally with the first sleeve and second sleeve.

In certain embodiments, the first sleeve and/or second sleeve comprises a magnetic core.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIGS. 1A-1B: Surgical instrument sleeve system according to an embodiment of the present disclosure with a pair of sleeves attached to a surgical instrument. In FIG. 1A, the surgical instrument is in an open position and, in FIG. 1B, the surgical instrument is in a closed position.

FIGS. 2A-2B: Side perspective views of each one of the sleeves in the surgical instrument sleeve system according to the embodiment shown in FIGS. 1A-1B. FIG. 2A is a side perspective view of a first sleeve and FIG. 2B is a side perspective view of a second sleeve.

FIGS. 3A-3B: Bottom views of each one of the sleeves in the surgical instrument sleeve system according to the embodiment shown in FIGS. 1A-1B. FIG. 3A is a bottom view of the first sleeve and FIG. 3B is a bottom view of the second sleeve.

FIGS. 4A-4B: Bottom views of a pair of sleeves in a surgical instrument sleeve system according to another embodiment of the present disclosure. FIG. 4A is a bottom view of a first sleeve containing a magnetic core and FIG. 4B is a bottom view of a second sleeve containing a magnetic core.

FIGS. 5A-5B: Perspective views of a surgical instrument sleeve system in which a pair of sleeves are interconnected by a bridging member according to an embodiment of the present disclosure. In FIG. 5A, the bridging member has a smooth engagement surface and, in FIG. 5B, the bridging member has a serrated engagement surface.

FIGS. 6A-6B: Surgical instrument sleeve system attached to a surgical instrument according to an embodiment of the present disclosure in which a pair of sleeves are interconnected by an integral bridging member. In FIG. 6A, the surgical instrument is in an open position and, in FIG. 6B, the surgical instrument is in a closed position.

FIG. 7: Surgical instrument sleeve system attached to a surgical instrument according to another embodiment of the present disclosure in which a pair of sleeves are interconnected by a non-integral bridging member.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this disclosure pertains.

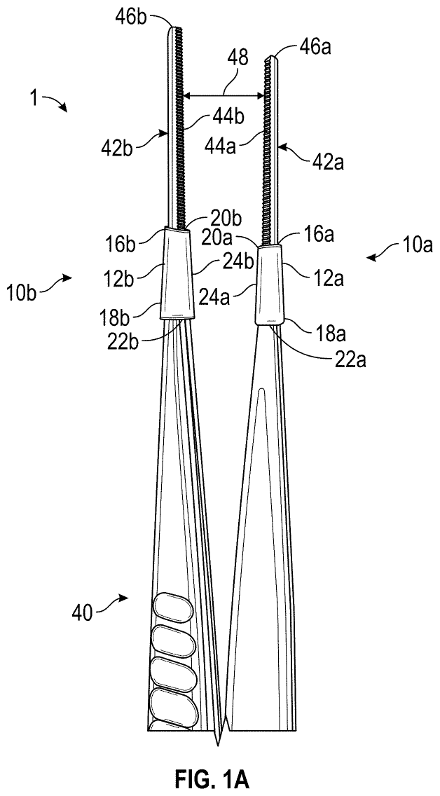

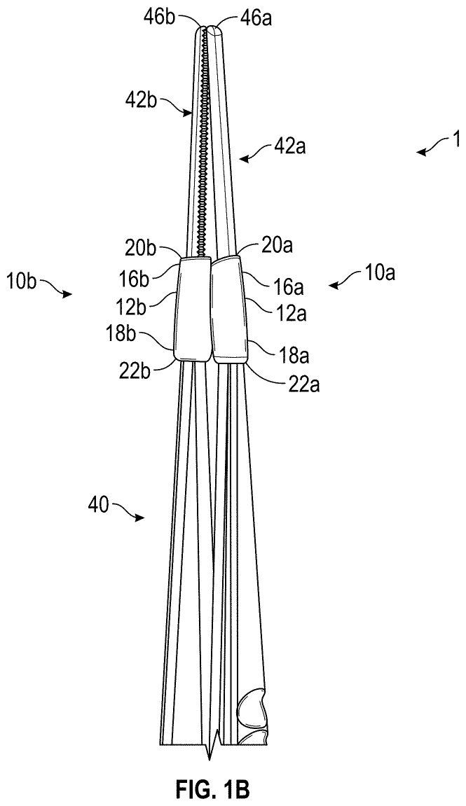

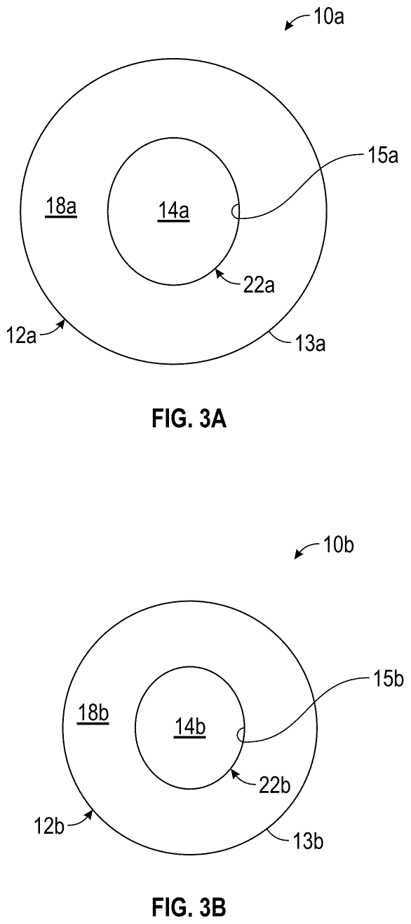

Provided herein is a system for overcoming at least some of the problems associated with handling suture material while using a surgical instrument such as forcepts. Referring now to the drawings, there is illustrated in FIGS. 1-7 a surgical instrument sleeve system, indicated generally at 1, in accordance with the present disclosure. The surgical instrument sleeve system 1 includes a pair of tubular sleeves 10 comprising a first sleeve 10a and a second sleeve 10b. Each sleeve 10a and 10b is adapted to attach to an opposing jaw 42a and 42b of a surgical instrument 40, such as forceps. As will be explained below, use of the surgical instrument sleeve system 1 may improve the ability of a surgical instrument to handle suture material and may broaden the capabilities of a surgeon's non-dominant hand during surgical procedures.

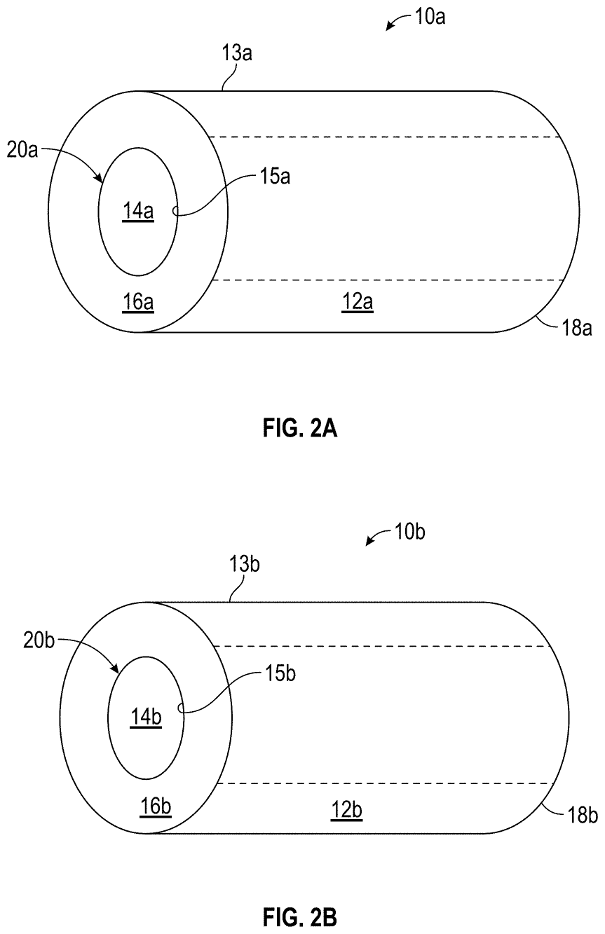

With continued reference to FIGS. 1-7, the first sleeve 10a can include a body 12a having a bore 14a extending within the body 12a between a first opening 20a at a first end 16a of the body 12a and a second opening 22a at a second end 18a of the body 12a. The first sleeve body 12a is defined by an outer wall 13a and an inner wall 15a. The outer wall 13a of the body 12a includes an engagement surface 24a, for a purpose that will be explained below. The inner wall 15a of the body 12a may define the bore 14a, as best illustrated in FIGS. 2, 3, and 5. The bore 14a may be positioned in the center of the body 12a, as best shown in the embodiments of FIGS. 2-5, but it is not required to be exactly in the center of the body 12a. As will be described in detail below, the bore 14a may have dimensions configured to accommodate the dimensions of a first jaw 42a of a surgical instrument 40.

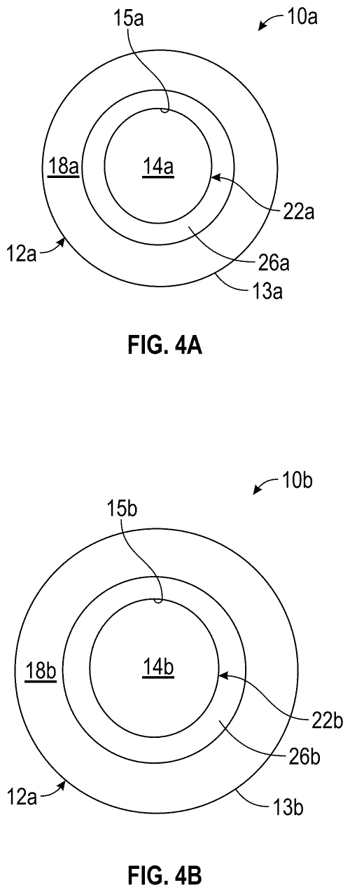

The first sleeve body 12a may have a tubular, or cylindrical, shape, but is not required to. For example, in the embodiments shown in FIGS. 2-5, the first sleeve 10a has a cylindrical body 12a. The body 12a may be formed from a surgical quality resilient material such as a medical-grade plastic, foam, rubber, or other synthetic material, and may have a soft, non-slippery outer surface. Also, the body 12a may be formed from a magnetic rubber or the body 12a may include a magnetic core 26a. In the embodiment shown in FIG. 4A, the first sleeve body 12a includes a magnetic core 26a positioned between the outer walls 13a of the body 12a and the bore 14a such that the magnetic core 26a defines the bore 14a. The magnetic core 26a may be concentric with the body 12a and/or the bore 14a, but does not need to be concentric with the body 12a or the bore 14a. The magnetic core 26a may be formed from a ferromagnetic material such as, but not limited to, Ni, Co, NiFe, CoFe, CoZrNb, NiFeCr, AlSiFe, NiFeRe, PtMn, TbFeCo, GdFeCo, TbFeCoZr, DyFeCo, GdDyFeCo, combinations thereof, and alloys thereof.

The second sleeve 10b can include a body 12b having a bore 14b extending within said body 12b between a first opening 20b at a first end 16b of the body 12b and a second opening 22b at a second end 18b of the body 12b. The second sleeve body 12b is defined by an outer wall 13b and an inner wall 15b. The outer wall 13b of the body 12b includes an engagement surface 24b, for a purpose that will be explained below. The inner wall 15b of the body 12b may define the bore 14b, as best illustrated in FIGS. 2, 3, and 5. The bore 14b may be positioned in the center of the body 12b, as best shown in the embodiments of FIGS. 2-5, but it is not required to be exactly in the center of the body 12b. As will be described in detail below, the bore 14b may have dimensions configured to accommodate the dimensions of a second jaw 42b of the surgical instrument 40.

The second sleeve body 12b may have a tubular, or cylindrical, shape, but is not required to. For example, in the embodiments shown in FIGS. 2-5, the second sleeve 10b has a cylindrical body 12b. The body 12b may be formed from a surgical quality resilient material such as a medical-grade plastic, foam, rubber, or other synthetic material, and may have a soft, non-slippery outer surface. Also, the body 12b may be formed from a magnetic rubber or the body 12b may include a magnetic core 26b. In the embodiment shown in FIG. 4B, the second sleeve body 12b includes a magnetic core 26b positioned between the outer walls 13b of the body 12b and the bore 14b such that the magnetic core 26b defines the bore 14b. The magnetic core 26b may be concentric with the body 12b and/or the bore 14b, but does not need to be concentric with the body 12b or the bore 14b. The magnetic core 26b may be formed from a ferromagnetic material such as, but not limited to, Ni, Co, NiFe, CoFe, CoZrNb, NiFeCr, AlSiFe, NiFeRe, PtMn, TbFeCo, GdFeCo, TbFeCoZr, DyFeCo, GdDyFeCo, combinations thereof, and alloys thereof. In certain embodiments, only one of the sleeves 12a, 12b includes a magnetic core 26a, 26b, and the other sleeve 12a, 12b does not include a magnetic core 26a, 26b. In other embodiments, neither of the sleeves 12a, 12b includes a magnetic core 26a, 26b.

In the embodiments shown in FIGS. 1-7, the first sleeve 10a and the second sleeve 10b are substantially similar to each other in terms of their dimensions and material composition. However, the first sleeve 10a and the second sleeve 10b may have different dimensions and may be formed from different materials.

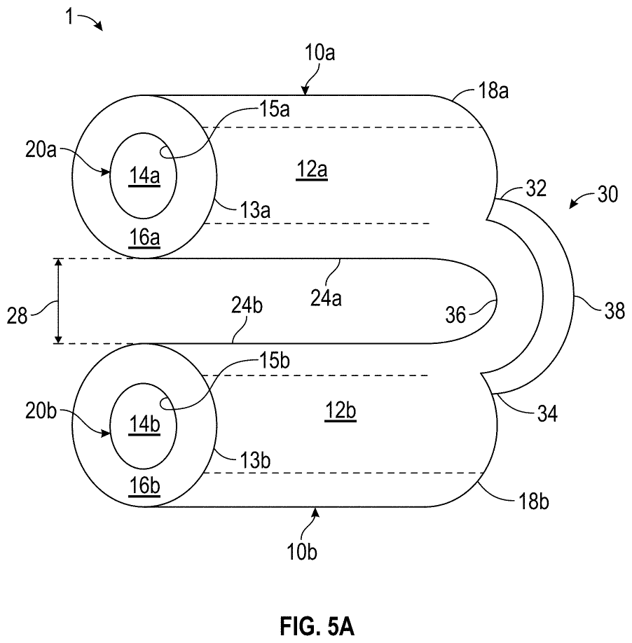

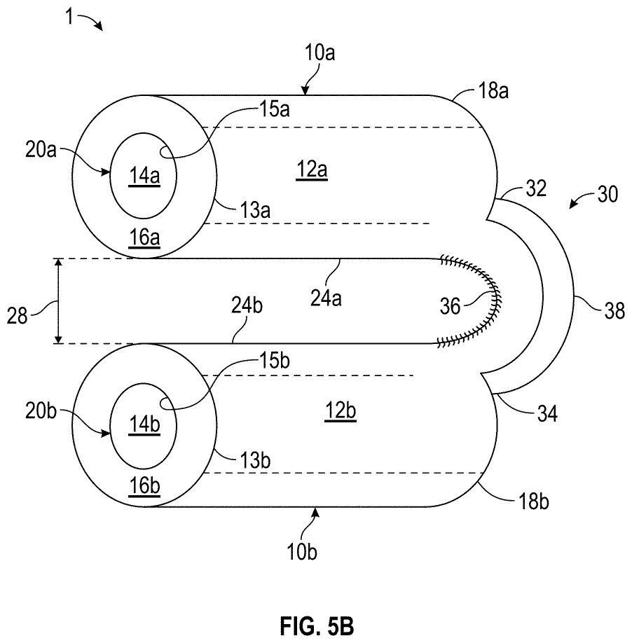

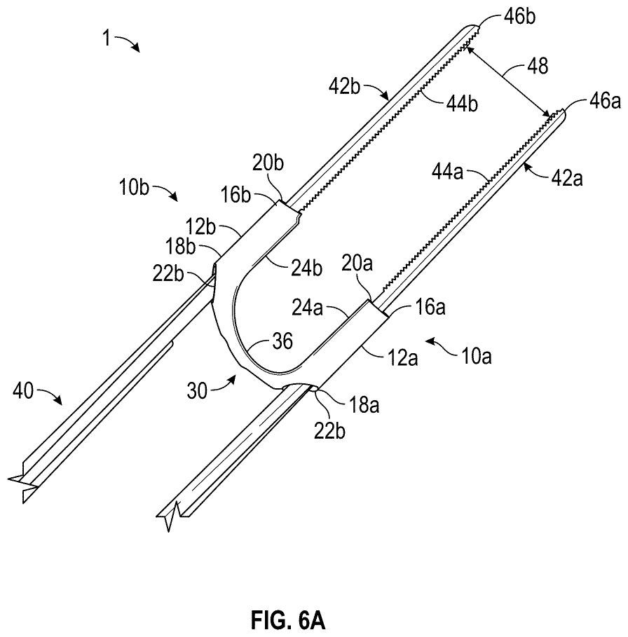

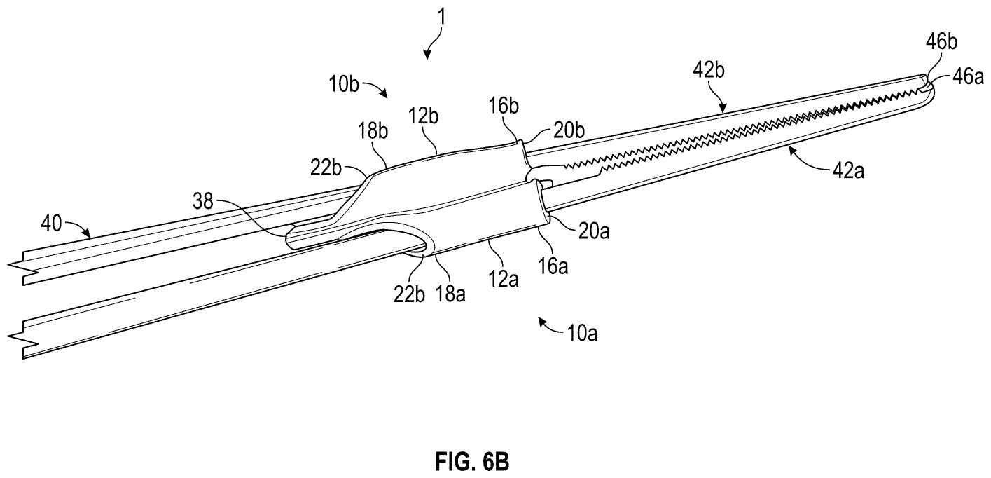

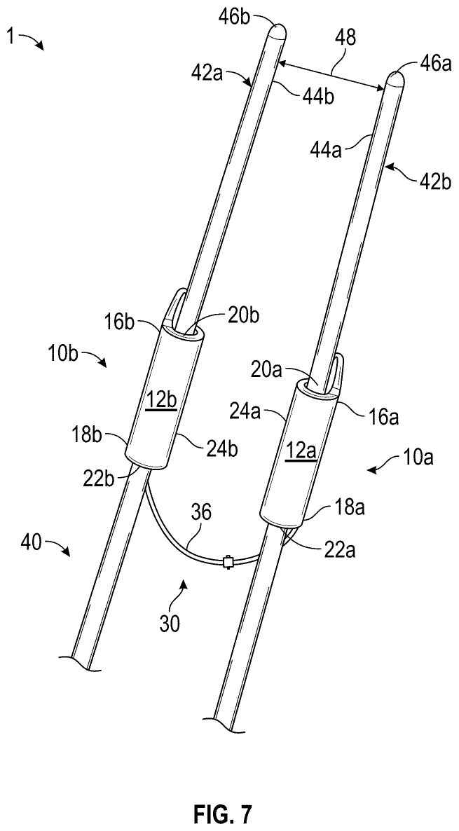

Referring now to FIGS. 5-7, the surgical instrument sleeve system 1 may further include a bridging member 30 that interconnects the first sleeve 10a and the second sleeve 10b. The bridging member 30 may be formed integrally with the first sleeve 10a and the second sleeve 10b, as shown in FIGS. 5 and 6, or the bridging member 30 may be a separate component that is connected between the first sleeve 10a and the second sleeve 10b, as shown in FIG. 7. The bridging member 30 may be formed from a flexible material such as a wire or any of the materials used to make the first sleeve body 12a and second sleeve body 12b described above.

The bridging member 30 includes a first end 32 connected to the first sleeve 10a and a second end 34 connected to the second sleeve 10b. As best illustrated in FIGS. 5A and 5B, the first end 32 of the bridging member 30 is connected to the first sleeve 10a at the second end 18a of the first sleeve body 12a and the second end 34 of the bridging member 30 is connected to the second sleeve 10b at the second end 18b of the second sleeve body 12b. However, in other embodiments, the bridging member 30 may be connected elsewhere on the sleeves 10a and 10b as long as the bridging member 30 does not prevent attachment of the sleeves 10a and 10b to a surgical instrument 40, for example, by obstructing access by the surgical instrument 40 to the first sleeve openings 20a and 22a and the second sleeve openings 20b and 22b.

The bridging member 30 includes an engagement surface 36 extending between the first end 32 of the bridging member 30 and the second end 34 of the bridging member 30. The engagement surface 36 is located on a portion of the bridging member 30 proximate to the first sleeve engagement surface 24a and the second sleeve engagement surface 24b, for a purpose that will be described below. The engagement surface 36 may be smooth, as shown in the embodiment of FIG. 5A, or serrated, as shown in the embodiment of FIG. 5B. In other embodiments, the bridging member engagement surface 36 may have cross-hatching, or other features configured to provide enhanced grip at the bridging member engagement surface 36.

The bridging member 30 may be bowed or curved, but it is not required to be bowed or curved. The bridging member 30 may also be pointed or flat, but it is not required to be pointed or flat. FIGS. 5-7 show an arcuate bridging member 30 that curves away from the sleeves 10a and 10b. This arcuate bridging member 30, combined with the attachment position of the bridging member 30 at the second ends 18a and 18b of the respective sleeves 1a and 10b, ensures that the bridging member 30, in use, does not intrude on the working space 28 between the first sleeve engagement surface 24a and the second sleeve engagement surface 24b when gripping a suture.

Referring now to FIGS. 1, 6, and 7, there is illustrated the use of the surgical instrument sleeve system 1 according to the present disclosure. FIGS. 1, 6, and 7 show a surgical instrument 40 fitted with the surgical instrument sleeve system 1. In these particular embodiments, the first and second sleeves 10a and 10b of the system 1 are attached to opposing jaws 42a and 42b of a pair of surgical forceps 40. The forceps 40 are used for illustration purposes only and it will be evident that other surgical instruments having similar jaw configurations can be used.

In use, the first sleeve 10a may be attached at a position on the first jaw 42a of the surgical instrument 40 such that a tip 46a of the first jaw 42a extends from the first opening 20a at the first end 16a of the body 12a. This configuration exposes a functional engagement surface 44a on the first jaw 42a which allows the surgical instrument 40 to retain its original function when the first sleeve 10a is attached to the surgical instrument 40. The first sleeve 10a may be retained on the surgical instrument 40 due to frictional engagement between the surfaces of the first jaw 42a and the inner walls 15a of the first sleeve body 12a when the first jaw 42a of the surgical instrument 40 extends within the bore 14a. Retention may also be assisted through magnetism if the first sleeve 10a contains a magnetic core 26a, as shown in FIG. 4A, and the first jaw 42a includes a magnetic material such as metal, or through some type of adhesive.

In use, the second sleeve 10b may be attached at a position on the second jaw 42b of the surgical instrument 40 such that a tip 46b of the second jaw 42b extends from the first opening 20b at the first end 16b of the body 12b. This configuration exposes a functional engagement surface 44b on the second jaw 42b which allows the surgical instrument 40 to retain its original function when the second sleeve 10b is attached to the surgical instrument 40. The second sleeve 10b may be retained on the surgical instrument 40 due to frictional engagement between the surfaces of the second jaw 42b and the inner walls 15b of the second sleeve body 12b when the second jaw 42b of the surgical instrument 40 extends within the bore 14b. Retention may also be assisted through magnetism if the second sleeve 10b contains a magnetic core 26b, as shown in FIG. 4B, and the second jaw 42b includes a magnetic material such as metal, or through some type of adhesive.

The first sleeve 10a and second sleeve 10b are positioned at a location on the opposing jaws 42a and 42b that permit an engagement surface 24a on the first sleeve 10a to cooperate with an engagement surface 24b on the second sleeve 10b when the surgical instrument 40 is closed, as best shown in FIGS. 1B and 6B. This cooperation between the first and second sleeves 10a and 10b permits the surgical instrument sleeve system 1 to handle delicate suture material. In addition, an engagement surface 44a of the first jaw 42a is still able to cooperate with an engagement surface 44b on the second jaw 42b when the surgical instrument 40 is in the closed position, as best shown in FIGS. 1B and 6B, to retain the original function of the surgical instrument 40 fitted with the surgical instrument sleeve system 1.

In summary, when the first and second sleeves 10a and 10b of the system 1 are properly positioned on the surgical instrument 40, the sleeves 10a and 10b allow direct manipulation of suture material while the surgical instrument 40 retains its original function. For example, the forceps 40 can be opened, as shown in FIGS. 1A and 6A, and the forceps 40 can be closed, as shown in FIGS. 1B and 6B, so that the functional engagement surfaces 44a and 44b on the opposing jaws 42a and 42b of the surgical instrument 40 cooperate with each other and the sleeve engagement surfaces 24a and 24b on the pair of sleeves 10a and 10b cooperate with each other.

Referring now to FIGS. 6 and 7, there is illustrated the use of a surgical instrument sleeve system 1 in which a bridging member 30 interconnects the first sleeve 10a and the second sleeve 10b. FIGS. 6 and 7 show a surgical instrument 40 fitted with the surgical instrument sleeve system 1 in which the sleeves 10a and 10b of the system 1 are attached to the opposing jaws 42a and 42b of a pair of surgical forceps 40, as described above. In use, the bridging member 30 may protect sutures from becoming trapped in a hinge of the surgical instrument 40, and may keep the suture material in close proximity to the sleeve engagement surfaces 24a and 24b.

The bridging member 30 should have a length sufficient to permit a surgical instrument 40 to operate in its normal manner when the surgical instrument sleeve system 1 is attached to the surgical instrument 40. For example, the bridging member 30 may have a length greater than or equal to the distance 48 between opposing jaws 42a and 42b to permit the jaws 42a and 42b to open without resistance from the bridging member 30. In FIGS. 6 and 7, the bridging member has a length greater than the jaw separation distance 48 which permits the surgical instrument 40 to completely open, as shown in FIGS. 6A and 7, and to close without interference, as shown FIG. 6B. The bridging member 30 should not have a length which causes interference with the opposing jaws 42a and 42b of the surgical instrument 40 when attempting to close the opposing jaws 42a and 42b of the surgical instrument 40. As best shown in FIG. 6B, the bridging member 30 should have the flexibility to fold, preferably at a midpoint 38 on the bridging member 30 between the sleeves 10a and 10b, with the bridging member 30 flexing towards the hinge of the instrument 40, and therefore away from the space between the engagement surfaces 24a and 24b of the sleeves 10a and 10b when in use.

The surgical instrument sleeve system 1 allows the surgeon to use a pair of opposing surgical instrument sleeves 10a and 10b to handle both the delicate suture material and the sharp tip of the surgical needle while still permitting the surgeon to use the functional engagement surfaces 44a and 44b of the surgical instrument 40 to manipulate tissue or to adjust the position of the surgical needle. And, if the surgical instrument sleeve system 1 includes the bridging member 30, the system 1 protects against sutures becoming trapped in the hinge of the surgical instrument 40. Moreover, the surgical instrument sleeve system 1 may include magnetic cores 26a and 26b which may be advantageous for handling magnetic sutures and in opposing the spring action of a surgical instrument 40 when the jaws 42a and 42b are moved towards a closed position.

The surgical instrument sleeve system 1 of the present disclosure can be simply, quickly, and securely attached to a surgical instrument 40. The surgical instrument sleeve system 1 may also be disposable. The surgical instrument sleeve system 1 improves the ability of a surgical instrument 40 to handle suture material and broadens the capabilities of the surgeon's non-dominant hand during surgical procedures. For example, the surgical instrument sleeve system 1 allows a surgeon to manipulate fine suture material during surgery without releasing the surgical instrument, such as forceps. This reduces, if not eliminates, the need for a skilled surgical assistant to be present during surgery to handle the suture material for the surgeon. Additionally, the surgical instrument sleeve system 1 permits the surgeon to manipulate sutures in deep body cavities where space may be limited and an assistant's hands may not readily reach. Lastly, the surgical instrument sleeve system 1 allows the surgeon to use the surfaces of the outer walls 13a and 13b of the sleeves 10a and 10b as a support mechanism to change the angle of the needle without damaging the tip of the surgical needle.

Certain embodiments of the systems and methods disclosed herein are defined above. It should be understood that the particular embodiments of the disclosure, are given by way of illustration only. From the above discussion, one skilled in the art can ascertain the essential characteristics of this disclosure, and without departing from the spirit and scope thereof, can make various changes and modifications to adapt the structure and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

what is claimed is:

1. A surgical instrument sleeve system comprising:
a first sleeve having a first sleeve body, wherein the first sleeve body includes a first sleeve bore extending within the first sleeve body between a first sleeve first opening at a first sleeve first end and a first sleeve second opening at a first sleeve second end;
a second sleeve having a second sleeve body, wherein the second sleeve body includes a second sleeve bore extending within the second sleeve body between a second sleeve first opening at a second sleeve first end and a second sleeve second opening at a second sleeve second end; and
a bridging member connecting the first sleeve and the second sleeve.

2. The surgical instrument sleeve system of claim 1, wherein the first sleeve body is cylindrical.

3. The surgical instrument sleeve system of claim 2, wherein the second sleeve body is cylindrical.

4. The surgical instrument sleeve system of claim 1, wherein the first sleeve further includes a first magnetic core positioned between the first sleeve body and the first sleeve bore.

5. The surgical instrument sleeve system of claim 4, wherein the second sleeve further includes a second magnetic core positioned between the second sleeve body and the second sleeve bore.

6. The surgical instrument sleeve system of claim 5, wherein the first magnetic core is concentric with the first sleeve body and the first sleeve bore and the second magnetic core is concentric with the second sleeve body and the second sleeve bore.

7. The surgical instrument sleeve system of claim 1, wherein the first sleeve and the second sleeve are formed from a surgical quality resilient material comprising medical-grade plastic, foam, or rubber.

8. The surgical instrument sleeve system of claim 1, wherein the bridging member is formed integrally with the first sleeve and second sleeve.

9. The surgical instrument sleeve system of claim 1, wherein the bridging member is arcuate.

10. The surgical instrument sleeve system of claim 1, wherein the bridging member includes a bridging member engagement surface, wherein the bridging member engagement surface is serrated.

11. The surgical instrument sleeve system of claim 1, wherein the bridging member has a bridging member first end attached to the first sleeve at the first sleeve second end and a bridging member second end attached to the second sleeve at the second sleeve second end.

12. A surgical instrument sleeve system comprising:
a first sleeve having a first sleeve body and a first magnetic core, wherein the first sleeve body includes a first sleeve bore extending within the first sleeve body between a first sleeve first opening at a first sleeve first end and a first sleeve second opening at a first sleeve second end, and wherein the first magnetic core is positioned between the first sleeve body and the first sleeve bore; and
a second sleeve having a second sleeve body and a second magnetic core, wherein the second sleeve body includes a second sleeve bore extending within the second sleeve body between a second sleeve first opening at a second sleeve first end and a second sleeve second opening at a second sleeve second end, and wherein the second magnetic core is positioned between the second sleeve body and the second sleeve bore.

13. The surgical instrument sleeve system of claim 12, wherein the first sleeve body is cylindrical.

14. The surgical instrument sleeve system of claim 13, wherein the second sleeve body is cylindrical.

15. The surgical instrument sleeve system of claim 12, wherein the first sleeve and the second sleeve are formed from a surgical quality resilient material comprising medical-grade plastic, foam, or rubber.

16. The surgical instrument sleeve system of claim 12, further including a bridging member connecting the first sleeve and the second sleeve.

17. The surgical instrument sleeve system of claim 16, wherein the bridging member includes a bridging member engagement surface, wherein the bridging member engagement surface is serrated.

18. The surgical instrument sleeve system of claim 16, wherein the bridging member is arcuate.

19. A method of using a surgical instrument sleeve system comprising:
attaching a first sleeve to a first jaw of a surgical instrument, wherein the first sleeve has a first sleeve body, and wherein the first sleeve body includes a first sleeve bore extending within the first sleeve body between a first sleeve first opening at a first sleeve first end and a first sleeve second opening at a first sleeve second end;
attaching a second sleeve to a second jaw of a surgical instrument, wherein the second sleeve has a second sleeve body, wherein the second sleeve body includes a second sleeve bore extending within the second sleeve body between a second sleeve first opening at a second sleeve first end and a second sleeve second opening at a second sleeve second end;
positioning the first sleeve at a location on the first jaw and the second sleeve at a location on the second jaw such that a first sleeve engagement surface on the first sleeve cooperates with a second sleeve engagement surface on the second sleeve; and
handling suture material between the first sleeve engagement surface and the second sleeve engagement surface.

20. The method of using the surgical instrument sleeve system of claim 19, wherein the first sleeve and the second sleeve are connected with a bridging member.
